(19) United States
(12) Patent Application Publication
(10) Pub. No.: US 2003/0118543 A1
(43) Pub. Date: Jun. 26, 2003

(54) Opaque conditioning composition

(75) Inventor: Daniel Raymond Pyles, Chicago, IL (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/243,223

(22) Filed: Sep. 13, 2002

Related U.S. Application Data
(63) Continuation of application No. 09/809,621, filed on Mar. 15, 2001.
Application No. 09/809,621 is a continuation of application No. 09/130,956, filed on Aug. 7, 1998, now abandoned.

(51) Int. Cl. A61K 7/075; A61K 7/08

(52) U.S. Cl. 424/70.28

(57) ABSTRACT

The present invention relates an opaque conditioner which comprises a monoalkyl quat from C16 to higher Carbon chain lengths (preferably C16 to C22) and a dialkyl quat from C16 to higher Carbon chain lengths (preferably C16 to C18). Also included is an amount of fatty alcohol necessary to opacify the conditioner. The monoalkyl quat is necessary in a ratio to the dialkyl quat of about not less than 2:1. The fatty alcohol is present in an amount from about 1% to about 4%.

### BACKGROUND OF THE INVENTION

Most individuals buy and use a hair shampoo for its cleansing properties. In addition to having clean hair, a consumer also desires sufficiently-conditioned hair that holds a preset configuration. However, hair shampoos generally are formulated with highly effective anionic surfactants that primarily clean as opposed to conditioning in the hair. Anionic surfactants not only remove the dirt and soil from the hair, but also remove sebum naturally present on the surface of the hair fibers. Therefore, the desirable cleansing properties of anionic surfactants also leave the hair in a cosmetically-unsatisfactory condition. Shampoos also do not detangle wet hair and do not impart residual conditioning benefits to dry hair, such as manageability or styleability of hair sets.

In general, shampoo compositions containing anionic surfactants, or nonionic surfactants or amphoteric surfactants, leave hair with an undesirable harsh, dull and dry touch, or feel, usually called “creak”, after the hair is shampooed and then rinsed with water. Furthermore, thoroughly cleansed hair also is extremely difficult to comb, in either the wet or the dry state, because the individual hair fibers tend to snarl, kink, and interlock with each other. In addition, incompletely dried hair, such as hair dried with a towel, has poor brushing properties, and after complete drying, the hair does not set well. The combing or brushing property of dry hair remains poor, and the hair has undesirable electrostatic properties in a low humidity atmosphere that causes the hair to “fly away”, thereby further reducing the brushing properties of the hair.

The unsatisfactory combing or brushing property of hair immediately after shampooing, or during trimming treatments after shampooing, also causes hair damage, such as split ends or hair breakage. In addition, the natural luster and resiliency of the hair is reduced. The overall unsatisfactory condition of shampooed hair often necessitates a subsequent post-shampoo treatment of the hair with a conditioning composition to improve these undesirable physical characteristics. Conditioning compositions typically are applied separately from the hair shampoo, and usually are rinses, cream-like emulsions or lotions containing a cationic compound.

Therefore, consumer needs traditionally have been met by the application of a shampoo to cleanse the hair, followed by the application of a conditioner composition to improve wet combing. The commonly accepted method has been to shampoo the hair, followed by rinsing the hair, and then separately applying a conditioner composition, followed by a second rinse. The wet combing problem has been solved by treating shampooed hair with a conditioner composition that coats the hair shaft and causes the individual hair shafts in a tress to resist tangling and matting because of the conditioner residue retained on the shaft.

However, the need for improved compositions that condition the hair, i.e., render the hair more manageable, has long been recognized in the art. As previously discussed, it is well-known that anionic surfactants are suitable for hair shampooing, and that cationic compounds, like cationic surfactants and cationic polymers, are useful as hair conditioners. Therefore, cationic compounds that are substantive to hair often are used to complete the hair cleansing and hair conditioning cycle.

The ability of cationic compounds to adsorb to or interact with the keratinous material of the hair makes these compounds desirable for improving wet hair detangling and dry hair manageability. However, cationic compounds that adsorb particularly strongly to the hair also can reduce the elasticity, body and set of the dried hair. Therefore, although conditioning compositions for application to freshly shampooed hair are well known, new and improved conditioning formulations based on cationic compounds are continually sought.

The following is a list of patents in this field.

JP 56169617 A

JP 56169615 A

JP 87008088 B

JP 56169614 A

JP 87008087 B

JP 56169613 A

JP 87008086 B

and U.S. Pat. No. 4,976,956.

The present invention is directed to a new opaque conditioning composition that is esthetically acceptable to consumers, improves the wet combing and dry combing properties of hair, and also leaves the dry hair with satisfactory cosmetic and physical properties, including, in particular, dry combing and feel, less hair coating, manageability, body, condition of the ends and set.

### SUMMARY OF THE INVENTION

The invention is an opaque conditioner that has a combination of two different types of conditioning agents and an emulsifier. The present invention is a low solids formulation that provides substantial conditioning benefit without compromising viscosity to users who use conditioners.

The purpose of the invention is to provide a conditioner with improved performance, while using effective materials at ratios that optimize their benefit.

The present invention relates to an opaque conditioner which comprises a monoalkyl quat from C16 to higher Carbon chain lengths (preferably C16 to C22) and a dialkyl quat each alkyl of which is independently from C16 to C18. Also included is an amount of fatty alcohol necessary to opacify the conditioner. The monoalkyl quat is necessary in a ratio to the dialkyl quat of about not less than 2:1. The fatty alcohol is present in an amount from about 1% to about 4%.

Another aspect of the invention is to provide a method of treating the hair to yield well-conditioned hair having esthetically pleasing physical properties by contacting the hair with an opaque conditioner of the present invention.

### DETAILED DESCRIPTION OF THE INVENTION

Unless indicated otherwise, as used herein, % means weight %.

The present invention relates to an opaque conditioner which comprises a monoalkyl quat from C16 to higher Carbon chain lengths (preferably C16 to C22) and a dialkyl quat from C16 to C18. Also included is an amount of fatty alcohol necessary to opacify the conditioner. The monoalkyl quat is necessary in a ratio to the dialkyl quat of about not less than 2:1. The ratio of monoalkyl quat to dialkyl quat can range from about 2:1 to about 5:1; or from about 2:1 to about 10:1; or from about 2:1 to about 20:1. Fatty alcohol is present in an amount from about 1% to about 4%.

Monoalkyl quats can be compounds of the formula N⁺R¹R²R³R⁴ X⁻ wherein R¹, R², and R³ are C1-C3 alkyl groups and R⁴ is a C16 or greater alkyl group; and X⁻ is chloride, bromide, methosulfate, ethosulfate, nitrate or tosylate.

Non-limiting examples of monoalkyl quats are:

cetyltrimethylammonium chloride (C16);

stearyltrimethylammonium chloride (C18);

behenetrimethylammonium chloride (C22);

cetrimonium bromide (C16);

soytrimonium chloride (C18);

tallowtrimonium chloride (C18);

behentrimethylammonium methosulfate (C22);

Peg-2 Olealmonium chloride (C18);

palmityltrimethylammonium chloride (C16);

hydrogenated tallowtrimethylammonium chloride (C18);

hydrogenated tallowtrimethylammonium bromide (C18);

hydrogenated tallowtrimethylammonium methosulfate (C18);

cetrimonium tosylate (C16): and

eicosyltrimethylammonium chloride (C20),

Dialkyl quats can be compounds of the formula N⁺R⁵R⁶R⁷R⁸ X⁻ wherein R⁵ and R⁶ are C1-C3 alkyl groups and R⁷and R⁸ are C16-C18 alkyl groups; and X⁻ is chloride, bromide, methosulfate, ethosulfate, nitrate, acetate, phosphate; or tosylate.

Non-limiting examples of dialkyl quats are:

dicetyidimethylammonium chloride(C16);

distearyldimethylammonium chloride (C18);

dipalmityldimethylammonium chloride (C16);

dihyrogenatedtallowdimethylammonium chloride (C18);

ditallowdimethylammonium chloride (C18)

dihyrogenatedtallowdimethylammonium bromide (C18)

dihyrogenatedtallowdimethylammonium methosulfate (C18)

The following are non-limiting examples of fatty alcohols which may be used in the compositions of the invention:

cetyl alcohol;

stearyl alcohol;

cetearyl alcohol;

behenyl alcohol; and

arachidyl alcohol.

Optional ingredients which may be included in the compositions of the invention are hydrocarbons such as paraffin, vaseline solid paraffin, squalene, oligomer olefins and the like; amidoamines such as stearamidopropyl dimethylamine, isostearamidoethyl morpholine, behenamidopropyl dimethylamine and the like; humectants such as glycerine, propylene glycol, glycerol, sorbitol and the like; esters, such as isopropyl palmitate, isopropyl myristate, and stearyl stearate and the like; emulsifiers such as glyceryl monostearate, sorbitan monopalmitate, polyoxyethylene stearate and the like; cellulose derivatives such as hydroxypropylcellulose; cationic cellulose, hydroxyethyl cellulose and the like; thickening agents such as natural polymers and the like; and other ingredients such as solvents, bacteriacides, colors, and fragrances.

Compositions of the invention may be prepared by methods which are known to those skilled in the art. Ingredients used in the preparation of compositions of the invention are either known or may be prepared by known methods.

Compositions of the invention are used to condition hair by first wetting the hair, applying the composition of the invention, lathering the hair, and then rinsing the hair. Alternatively, water and conditioner may be applied to the hair simultaneously. Conditioning with compositions may be done right after shampooing when the hair is still wet. Alternatively, conditioning the hair may be done separately from shampooing.

Compositions of the invention provide unexpectedly superior conditioning benefits when compared with prior art formulations. Compositions of the invention provide unexpectedly provide a high, consumer acceptable viscosity using relatively low levels of monoalkyl quat, dialkyl quat, and fatty alcohol.

Finally, compositions of the invention provide unexpectedly superior conditioning without the use of increased fatty alcohols.

To demonstrate the new and unexpected results achieved by the present invention, the following compositions were prepared.

Compositions of the present invention have significantly more conditioning versus a formulation with ingredients that fall outside of the ratios set by the present invention. The following chart illustrates.

A.) Conditioning Performance

Instron Wet Combing Test

| | Composition | Compo- | Compo- |
|---|---|---|---|
| | (A) | sition | sition |
| Ingredients | Prior Art | (B) | (C) |
| Natrosol 250HHR, 97% active | .30 | — | — |
| Cetrimonium Chloride, 30% active | 1.2 | 2.15 | 2.8 |
| Isopropyl Palmitate, 100% active | .5 | — | — |
| Distearyldimonium Chloride, 95% | 1.1 | .20 | .1 |
| active | | | |
| Stearyl Alcohol, 100% active | — | .50 | — |
| Cetearyl Alcohol, 100% active | 1.9 | 2.5 | — |
| Cetyl Alcohol, 100% active | — | — | 3.0 |
| Other¹ | q.s. | q.s. | q.s. |
| Combing Force (gm force) | 14.1 | 10.0A | 7.9AB |

Other¹ - soft water, fragrance, preservatives, and other minor ingredients.

The combing force shows that:

Composition (C), with a monoalkyl:dialkyl ratio of 8.8:1 is significantly better than Composition (B), with a monoalkyl:dialkyl ratio of 3.4:1. Composition (C ) with a mono:dialkyl ratio of 8.8:1 is significantly better than that Composition (A) with a monoalkyl:dialkyl ratio of 0.345:1. Composition (B), with a monoalkyl:dialkyl ratio of 3.4:1 is significantly better than Composition (A) with a monoalkyl:dialkyl ratio of 0.345:1.

The following chart summarizes consumer preference for a composition of the invention as opposed to a composition of the prior art.

| | Composition | |
|---|---|---|
| | (A) | Composition |
| Ingredients | Prior Art | (D) |
| Natrosol 250HHR, 97% active | .30 | — |
| Cetrimonium Chloride, 30% active | 1.2 | 2.8 |
| Isopropyl Palmitate, 100% active | .50 | — |
| Distearyldimonium Chloride, 95% active | 1.1 | .15 |
| Cetearyl Alcohol, 100% active | 1.9 | — |
| Cetyl Alcohol, 100% active | — | 3.0 |
| Other² | q.s. | q.s. |
| Conditions your hair | 16 | 40A |
| Adds moisture to your hair | 14 | 32A |
| Gives your hair body | 16 | 32A |
| Leaves hair easy to comb wet | 14 | 30A |
| Leaves hair easy to comb dry | 8 | 22A |
| Consistency | 9 | 23A |

Other² - soft water, fragrance, preservatives, and other minor ingredients.

This chart shows that consistent with the Instron Study consumers rate the Invention, composition (D) as a better wet combing conditioner as well as rating the invention higher on many key conditioning attributes. Composition (D) with a monoalkyl:dialkyl ratio of 5.9:1 is significantly preferred over Composition (A) with a monoalkyl:dialkyl ratio of 0.345:1.

The consumer ratings given just above mean the following: A letter next to a rating signifies that the product measured is significantly different than the product designated by the letter at a 90% C.I.

Evaluation of 2:1 versus 1:1 mono:dialkyl quat ratio

| | Composition | Composition |
|---|---|---|
| Ingredients | (E) | (F) |
| Cetrimonium Chloride, 30% active | 2.22 | 1.7 |
| Distearyldimonium Chloride, 95% active | .33 | .50 |
| Cetyl Alcohol, 100% active | 3.0 | 3.0 |
| Other³ | | |
| Ratio mono:di | 2:1 | 1:1 |
| salon blitz consumer evaluations | | |
| like conditioning | 6.58 | 5.75 |
| like fullness/volume | 6.58F | 5.25 |
| Like bounce | 6.58F | 5.17 |
| ease of combing wet | 8.33 | 7.50 |
| ease of combing dry | 8.42 | 7.42 |
| Moisturized | 7.25F | 6.00 |
| Fullness | 6.00F | 4.58 |

Other³ - soft water, fragrance, preservatives, and other minor ingredients.

Consumers evaluated products and filled out a questionnaire regarding their responses to the product. There were approximately 15 consumers for each product. A letter next to a rating signifies that the product measured is significantly different than the product designated by the letter at a 90% C.I. From the above table it can be seen that a product with a 2:1 mono:dialkyl quat ratio was significantly better in the salon blitz evaluations than a product with a 1:1 mono:dialkyl quat ratio.

B.) Viscosity

An acceptable and preferred viscosity is achieved by optimizing the levels of the three ingredients, monoalkyl quat, dialkyl quat and fatty alcohol. Small levels of the dialkyl quat give a significant increase in viscosity, thus allowing a lower level of fatty alcohol. (When fatty alcohol is at higher levels it can affect the performance of the quats.) The fatty alcohol can be as low as 1 or 2% as long as there is a small presence of the dialkyl quat. However without the dialkyl quat, 1 or 2% fatty alcohol results in an unacceptable viscosity. The following charts illustrate this.

Effect of Dialkyl quat on low fatty alcohol systems

| | Compo- | Compo- | Compo- | Compo- | Compo- |
|---|---|---|---|---|---|
| | sition | sition | sition | sition | sition |
| Ingredients | (G) | (H) | (I) | (J) | (D) |
| Cetrimonium Chloride, | 2.8 | 2.8 | 2.8 | 2.8 | 2.8 |
| 30% active | | | | | |
| Distearyldimonium | — | — | .15 | — | 15 |
| Chloride, 95% active | | | | | |
| Cetyl Alcohol, 100% | 1.0 | 2.0 | 2.0 | 3.0 | 3.0 |
| active | | | | | |
| Other⁴ | q.s. | q.s. | q.s. | q.s. | q.s. |
| Viscosity TA/.5. RV | 8k, 470 | 46k, | 116k, | 74k, | 128k, |
| 4/20 @ 7 days room | | 2200 | 6400 | 4600 | 6600 |
| temperature (cps) | | | | | |

Other⁴ - soft water, fragrance, preservatives, and other minor ingredients.

The above chart shows that the addition of even a small amount of the Dialkyl quat, 0.15%, results in a significant increase in viscosity. The following chart illustrates that increasing the fatty alcohol without dialkyl quat will not give you substantially more viscosity. Viscosity TA/0.5. and RV 4/20@7 days are Brookfield measures of viscosity. TA/0.5. is a low shear measure with TA spindle at 0.5 rpm. RV 4/20 is a high shear measure which uses the RV/4 spindle at 20 rpm.

Effect of Increased fatty alcohol on systems with no Dialkyl quat.

| | Compo- | Compo- | Compo- | Compo- | Compo- |
|---|---|---|---|---|---|
| | sition | sition | sition | sition | sition |
| Ingredients | (H) | (J) | (K) | (L) | (D) |
| Cetrimonium Chloride, | 2.8 | 2.8 | 2.8 | 2.8 | 2.8 |
| 30% active | | | | | |
| Distearyldimonium | — | — | — | — | .15 |
| Chloride, 95% active | | | | | |
| Cetyl Alcohol, 100% | 2.0 | 3.0 | 4.0 | 5.0 | 3.0 |
| active | | | | | |
| Other⁵ | q.s. | q.s. | q.s. | q.s. | q.s. |
| Viscosity TA/.5. RV | 46k, | 74k, | 76k, | 78k, | 128k, |
| 4/20 @ 7 days | 2200 | 4600 | 4900 | 5000 | 6600 |

Other⁵ - soft water, fragrance, preservatives, and other minor ingredients.

Composition (D) with only 3% fatty alcohol is significantly more viscous than Composition (K) or Composition (L) with higher levels of fatty alcohol. Other monoalkyl quats such as Behentrimonium Chloride and other Dialkylquats such as Dicetyldimonium Chloride are also effective. The following chart illustrates this.

Effect of other quats

| | Composition | Composition |
|---|---|---|
| Ingredients | (M) | (N) |
| Behentrimonium Chloride, 80% active | 1.05 | 1.05 |
| Distearyldimonium Chloride, 95% active | .15 | — |
| Cetyl Alcohol, 100% active | 3.0 | 3.0 |
| Other⁶ | q.s. | q.s. |
| Viscosity TA/.5. RV 4/20 @ 7 days (cps) | 90k, 5400 | 62k, 3400 |

Other⁶ - soft water, fragrance, preservatives, and other minor ingredients.

The dialkyl quat also enhances the viscosity when Behentrimonium Chloride is used in place of Cetrimonium Chloride.

C.) Deposition

Fatty Alcohol and Quat Deposition

| | Composition | Composition |
|---|---|---|
| Ingredients | (D) | (J) |
| Cetrimonium Chloride, 30% active | 2.8 | 2.8 |
| Distearyldimonium Chloride, 95% active | .15 | — |
| Cetyl Alcohol, 100% active | 3.0 | 3.0 |
| Other⁷ | q.s. | q.s. |
| Fatty alcohol deposition | 450 | 305 |
| (ug/g of hair +/− 10%) | | |
| Cetrimonium Chloride deposition | 100 | 50 |
| (ppm of hair, values are +/− 25%) | | |
| Distearyldimonium Chloride deposition | 10 | not detected |
| (ppm of hair, values are +/− 25%) | | |

Other⁷ - soft water, fragrance, preservatives, and other minor ingredients.

Duplicate tresses were extracted twice with 50 ml portions of chloroform. The extracts were combined and concentrated. Fatty alcohols were measures by GC-MS relative to an internal standard and measured relative response factors were used. Quats were measured on 1:100 dilutions by electrospray LC-MS.

Sample (D), which in addition to its monoalkyl quat has a small amount of dialkyl quat, surprisingly and unexpectedly deposited more fatty alcohols and quats on the tresses than did sample (J) which lacked dialkyl quat. The use of monoalkyl quat in combination with dialkyl quat surprisingly and unexpectedly increases the deposition of fatty alcohols and quats on hair and thus increases the hair conditioning benefits of compositions of the invention.

D.) Phase Behavior

Conditioners are believed to perform best when they are formulated in the liquid crystalline phase. Dialkyl quats are generally thought to be in the liquid crystalline phase. We have found that monoalkyl quats, such as, Cetyl Trimethylammonium Chloride (CTAC) need increased amounts of fatty alcohol to move the CTAC in the liquid crystalline phase. It is believed that the present invention does not use increased amounts of fatty alcohol to achieve a conditioner in the liquid crystalline phase

What is claimed is:

1. An opaque hair conditioning composition comprising:
(a) a monoalkyl quat having 16 or greater carbon atoms in an alkyl substituent;
(b) a dialkyl quat having 16 to 18 carbon atoms in each alkyl substituent and
(c) a fatty alcohol in an amount sufficient to opacify said composition;
wherein the ratio of (a) to (b) is at least 2:1.

2. A composition according to claim 1 wherein the ratio of (a) to (b) is about 2:1 to about 20:1.

3. A composition according to claim 2 wherein the ratio of (a) to (b) is about 2:1 to about 10:1.

4. A composition according to claim 3 wherein the ratio of (a) to (b) is about 2:1 to about 5:1.

5. A composition in accordance with claim 1 wherein said fatty alcohol is present at about 1 to about 4%.

6. A composition in accordance with claim 1 wherein said monoalkyl quat is selected from the group consisting of behentrimonium chloride and cetrimonium chloride.

7. A composition in accordance with claim 1 wherein said dialkyl quat is selected from the group consisting of dicetyldimonium chloride and distearyldimonium chloride.

8. A composition in accordance with claim 1 wherein said monoalkyl quat is cetrimonium chloride.

9. A composition in accordance with claim 1 wherein said dialkyl quat is distearyldimonium chloride.

10. A composition in accordance with claim 1 wherein said fatty alcohol is cetyl alcohol.

11. A method for conditioning hair which comprises contacting hair with a composition of claim 1.
